(19) United States
(12) Patent Application Publication
(10) Pub. No.: US 2005/0171086 A1
(43) Pub. Date: Aug. 4, 2005

(54) Compositions for treating CNS disorders

(75) Inventors: Michael A. Brodney, East Lyme, CT (US); Harry R. Howard JR., Bristol, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/048,013

(22) Filed: Jan. 28, 2005

Related U.S. Application Data
(60) Provisional application No. 60/539,939, filed on Jan. 29, 2004.

(51) Int. Cl. A61K 31/55; A61K 31/5377; A61K 31/498

(52) U.S. Cl. 514/210.21; 514/249; 514/217.05; 514/234.2

(57) ABSTRACT

An aminomethylpyridyloxymethyl/benzisoxazole substituted azabicyclic compound, a pharmaceutical composition comprising same, and a method of treating one or more CNS or other disorders, including concurrent treatment of disorders such as schizophrenia and depression.

This application claims priority under 35 U.S.C 119 of U.S. Provisional 60/539,939 filed Jan. 29, 2004. The entire contents of the prior application are incorporated herein by reference.

### FIELD OF THE INVENTION

The invention pertains to a pharmaceutical composition comprising aminomethylpyridyloxymethyl/benzisoxazole substituted azabicyclic compounds that, among other things, serves as an effective 5-HT1B, 5-HT2A and D2 receptor inhibitor, e.g. antagonist, inverse agonist and/or partial agonist in combination with atypical antipsychotics. The invention also relates to the use of said pharmaceutical composition for treating CNS disorders.

### BACKGROUND OF THE INVENTION

Disorders of the Central Nervous System (CNS) can be medically treated in various ways. Of increasing importance in this regard are psychotropic drugs. But while such drugs have therapeutic effects, they also may cause unwanted and serious side effects. For example, schizophrenia can be treated with so-called typical antipsychotic drugs, which have been theorized to block certain dopamine (D2) receptors in the brain thought responsible for the positive symptoms of delusions, disordered thinking and the like. However, while these drugs can ameliorate some of the positive symptoms, they can also adversely affect the motor system, causing involuntary movement disorders and muscle problems such as spasms, cramps, tremors and Parkinsonism. These types of side effects—generally characterized as Extrapyramidal Symptoms (EPS)—can be severe enough to disrupt daily activities.

The situation is further complicated when several CNS disorders are present in a patient. For example, psychosis, such as schizophrenia, can often co-exist with depression, anxiety, obsessive-compulsive disorder (OCD) and other such illnesses. In such cases, treatment often entails the administration of a combination of drugs, e.g., one to treat schizophrenia and one to treat depression or other co-morbid CNS ailments. Because each such drug has its own side effects, the combined administration can lead to a multiplication or enhancement of same, all to the detriment of the patient. Moreover, it is theorized that a different brain receptor, or combination or permutation of receptors, are somehow implicated in each of the various CNS disorders; for example, schizophrenia has been thought to involve D2 and 5HT-2A receptors whereas depression has been associated with 5HT-1B receptors. A class of aminomethylphenoxymethyl/benzisoxazole substituted azabicyclic compounds, useful as selective agonists and antagonists of serotonin 1 (5-HT1) receptors, is described in WO 99/52907 to Bright.

It has hitherto proven difficult to find a medicament that can treat a patient suffering from diverse CNS disorders where a plurality of different receptors are in play. Accordingly, there is an on-going need for a pharmaceutical composition that has a pronounced reduction in side effects, and that can treat multiple CNS disorders in which an antagonist or agonist to different receptors is indicated. Specifically, it would be desirable to find a pharmaceutical composition that can concurrently treat schizophrenia and depression in which D2, 5HT-2A and 5HT-1B receptors are involved.

U.S. Patent Application having U.S. patent application Ser. No. 60/453,925 filed Mar. 12, 2003, entitled “Pyridyloxymethyl and Benzisooxazole Azabicyclic Derivatives” and owned by the assignee, discloses in part, compounds of the formula 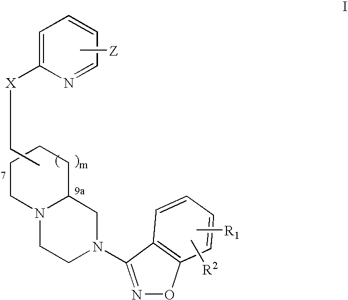
or the (R) or (S) enantiomer thereof, or the cis or trans isomer thereof, or a pharmaceutically acceptable salt, solvate or prodrug thereof, or of any of the foregoing, wherein m is 0 or 1; Z is 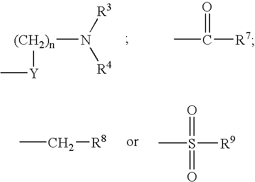
wherein R⁷ is hydrogen or (C₁-C₃)alkoxy; R⁸ is hydrogen, hydroxy, or (C₁-C₃)alkoxy;

and R⁹ is (C₁-C₃)alkoxy;

X is oxygen or NR, wherein R is hydrogen or (C₁-C₆)alkyl;

Y is methylene, wherein n is 0, 1 or 2; or oxygen, nitrogen or sulfur, wherein n is 2, 3 or 4;

R¹ and R² are each independently hydrogen, halogen, or a (C₁-C₆)alkyl, (C₁-C₆)alkoxy or a (C₁-C₆)alkoxy(C₁-C₆)alkyl group, any one of which groups may be unsubstituted or substituted with one or more halogens;

R³ and R⁴ are each independently hydrogen, a (C₁-C₆)alkyl, a (C₃-C₇)cycloalkyl, or a 5 to 6 membered heterocyclic group, any one of which groups may be unsubstituted or substituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups; or

R³ and R⁴ together with the nitrogen atom to which they are attached form:

a 3 to 7 membered optionally unsaturated monocyclic ring; or

a 4 to 10 membered optionally unsaturated polycyclic ring, wherein said monocyclic or polycyclic ring optionally has one or two additional heteroatoms selected from nitrogen, oxygen and sulfur,

wherein any of said rings (i) or (ii) may be unsubstituted or substituted with one or more (C₁-C₄)alkyl, (C₁-C₄)alkoxy, (C₁-C₄)alkoxy(C₁-C₄)alkyl, (C₃-C₇)cycloalkyl, (C₆-C₁₀)aryl, (C₇ to C₁₃)aralkyl, a 5 to 10 membered heteroaryl, hydroxy, amino, cyano, or halogen groups.

It discloses that these compounds are useful for treating CNS disorders. The present invention represents a second generation product and is an improvement over the pharmaceutical compound described in the aforementioned patent application

More specifically, the present invention is directed to combinations of the compound of Formula I with atypical antipsychotics.

### SUMMARY OF THE INVENTION

The present invention addresses the aforesaid needs. In one aspect, the invention relates to a pharmaceutical composition comprising a first component which is a pharmaceutically effective amount of the combination of an atypical antipsychotic compound and a second component which is denoted herein as Formula I, and a pharmaceutical carrier therefore the compound of Formula I having the formula: 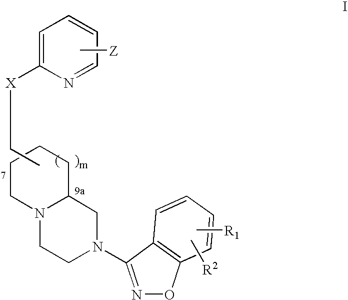
or the (R) or (S) enantiomer thereof, or other stereoisomers thereof, or a pharmaceutically acceptable salt, solvate or prodrug thereof or of any of the foregoing, wherein m is 0 or 1; Z is 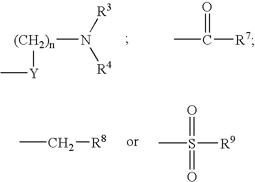
wherein R⁷ is hydrogen or (C₁-C₃)alkoxy; R⁸ is hydrogen, hydroxy, or (C₁-C₃)alkoxy;

and R⁹ is (C₁-C₃)alkoxy;

X is oxygen or NR, wherein R is hydrogen or (C₁-C₆)alkyl;

Y is methylene, wherein n is 0, 1 or 2; or oxygen, nitrogen or sulfur, wherein n is 2, 3 or 4;

R¹ and R² are each independently hydrogen, halogen, or a (C₁-C₆)alkyl, (C₁-C₆)alkoxy or a (C₁-C₆)alkoxy(C₁-C₆)alkyl group, any one of which groups may be unsubstituted or substituted with one or more halogens;

R³ and R⁴ are each independently hydrogen, a (C₁-C₆)alkyl, a (C₃-C₇)cycloalkyl, or a 5 to 6 membered heterocyclic group, any one of which groups may be unsubstituted or substituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups; or

R³ and R⁴ together with the nitrogen atom to which they are attached form:

a 3 to 7 membered optionally unsaturated monocyclic ring; or

a 4 to 10 membered optionally unsaturated polycyclic ring, wherein said monocyclic or polycyclic ring optionally has one or two additional heteroatoms selected from nitrogen, oxygen and sulfur,

wherein any of said rings (i) or (ii) may be unsubstituted or substituted with one or more (C₁-C₄)alkyl, (C₁-C₄)alkoxy, (C₁-C₄)alkoxy(C₁-C₄)alkyl, (C₃-C₇)cycloalkyl, (C₆-C₁₀)aryl, (C₇ to C₁₃)aralkyl, a 5 to 10 membered heteroaryl, hydroxy, amino, cyano, or halogen groups.

Another aspect of the present invention is directed to a method of treating one or more CNS disorders in a mammal in need of such treatment by administering thereto the pharmaceutical composition described hereinabove in an amount effective to treat such CNS disorder.

Another aspect of the present invention is directed to a kit containing the combination of a compound of Formula I with an atypical antipsychotic, optionally with instructions for use. The compound of Formula I and the atypical antipsychotic compound may either be admixed together in the kit with a pharmaceutical carrier or they may each be in separate compartments within a container. In the latter case, one of the aforementioned components may be admixed together with a pharmaceutical carrier or each may be admixed with a pharmaceutical carrier in separate compartments.

### DETAILED DESCRIPTION OF THE INVENTION

As described hereinabove, an embodiment of the present invention is directed to a composition comprising the combination of an atypical antipsychotic compound as the first component and a compound of Formula I as the second component.

The first component is a compound which acts as an atypical antipsychotic. The atypical antipsychotic, when present in therapeutically effective amounts reduces incidents of EPS. In addition, the atypical antipsychotic can alleviate not only some of the positive symptoms of CNS disorders, such as schizophrenia, but some of the negative symptoms as well, such as emotional unresponsiveness, social withdrawal and the like.

The atypical antipsychotic is a term of art well understood by one of ordinary skill. Typically it exhibits a different and recognizable clinical and pharmacological profile relative to a conventional antipsychotic and exhibits advantages over the conventional antipsychotics. The conventional antipsychotics, such as haloperidol are selective antagonists of dopamine (D2) receptors. The atypical antipsychotics also have D2 antagonist properties, but their binding kinetics to those receptors are different and the antagonist activity to those receptors are relatively weak. However, in addition, they have activity at other receptors, such as 5HT_{2A}, 5HT_{2c} and 5HT_{1d}. The essential feature of an atypical antipsychotic is that it exhibits less acute extrapyramidol symptoms, especially dystonias, associated with therapy as compared to the conventional antipsychotic. For example, atypical antipsychotics have greater efficacy in the treatment of overall psychotherapy in schizophrenics, nonresponsive to typical antipsychotics; (2) greater efficacy in the treatment of negative symptoms of schizophrenia; (3) less frequent and quantitatively smaller increase in serum prolactin concentrations associated with therapy; (4) lower risks of EPS or TD (tardive dyskinesia); and (5) improved cognitive functions. See, e.g., Beasley, et al. Neuropsychopharmacology, 14(2): 111, (1996).

Examples of atypical antipsychotics which can be used in the present invention include but are not limited to asenapine olanzapine, clozapine, risperidone, sertindole, quetiapine, aripiperazole, amisulpride, ziprasidone, mirtazapine and the like.

Ziprasidone, 5-[2[-4-(1,2-benzisothiazol-3-yl)piperazin-1-yl])ethyl]-6-chloro-1,3-dihydro-2H-indol-2-one hydrochloride, is an atypical antipsychotic having in vitro activity as a 5HT_{1A} receptor antagonist, a 5HT_{2A} and dopamine D2 receptor antagonist, and an inhibitor of serotonin and norepinephrine uptake. It is described in U.S. Pat. Nos. 4,831,031, 5,312,295, 6,387,904, 6,245,765, and 6,245,766 and European Patent application EP901781, published Mar. 17, 1999, the contents of all of which are incorporated herein by reference. It is efficacious in the treatment of patients with schizophrenia, affective and anxiety symptoms associated with schizoaffective disorder and bipolar disorder.

Olanzapine, which is 2-methyl-4-(4-methyl-1-piperazinyl)-10H-thieno[2,3-b][1,5]benzodiazepine, is described in U.S. Pat. No. 5,229,382, the contents of which are incorporated by reference. It is also described as being useful for the treatment of schizophrenia, schizophreniform disorder, acute mania, mild anxiety states and psychosis.

Clozapine, 8-chloro-11-(4-methyl-1-piperazinyl)-5H-dibenzo [b,e][1,4]diazepine is shown to have clinical efficacy in the treatment of schizophrenia. See, Hanes et al., Psychopharmacol Bull. 24, 62 (1998). It is also described in U.S. Pat. No. 3,539,573, the contents of which are incorporated by reference in its entirety.

Risperidone is 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one. Risperidone and its use in the treatment of psychotic diseases are described in U.S. Pat. No. 4,804,663 which is herein incorporated by reference in its entirety;

Sertindole is 1-[2-[4-[5-chloro-1-(4-fluorophenyl)-1H-indol-3-yl]-1-piperidinyl]ethyl]limidazolidin-2-one. Sertindole is described in U.S. Pat. No. 4,710,500, and its use in the treatment of schizophrenia is described in U.S. Pat. Nos. 5,112,838 and 5,238,945, the contents of all of which are herein incorporated by reference in their entirety;

Quetiapine is 2-[2-(4-dibenzo[b,f][1,4]thiazepin-11-yl-1-piperazinyl)ethoxy]ethanol. Quetiapine and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt;

Aripiprazole, 7-{4-[4-(2,3-dichlorophenyl)-1-piperazinyl-butoxy}-3-, 4-dihydro carbostyril or 7-{4-[4-(2,3-dichlorophenyl)-1piperazinyl]-butoxy}-3,4-dihydro-2(1H)-quinolinone, is an atypical antipsychotic agent used for the treatment of schizophrenia and is described in U.S. Pat. Nos. 4,734,416 and 5,006,528 both of which are herein incorporated by reference in their entirety;

Amisulpride, which is 4-amino-N-[1-ethyl-2-pyrrolidinyl)methyl]-5-(ethylsulfonyl)-2-methoxybenzamide is a known antipsychotic. It exhibits dopamine antagonist activity in rats. See P. Protais, et al. Neuropharmacol, 24, 861 (1985). It is described in U.S. Pat. No. 4,401,822, the contents of which are incorporated by reference; and

Mirtazepine, which is 1, 2, 3, 4, 10, 14b-hexa-hydro-2-methyl pyrazino[2, 1-a]pyrido[2,3-c]-[2]benzazepine is useful for treatment of major depressive disorders. It is described in U.S. Pat. No. 4,062,848, the contents of which are incorporated by reference.

Asenapine, trans-5-chloro-2-methyl-2,3,3a, 12b-tetrahydro-1H-dibenz[2,3:6,7]oxepino[4,5-c]pyrrole. Preparation and use of asenapine is described in U.S. Pat. Nos. 4,145,434 and 5,763,476.

The most preferred atypical antipsychotic is ziprasidone.

The second component is a compound having Formula I described hereinabove. The second component is described in copending application having serial number U.S. Ser. No. 60/453,925 filed Mar. 12, 2003, the contents of which are incorporated by reference. The compounds of Formula I exhibit, inter alia, binding activity to one or multiple receptors, including D2, 5HT1B and 5HT2A receptors, individually or in any combination thereof. The compounds are inhibitors of activity at D2, 5HT1B and 5HT2A receptors. In a preferred embodiment, the compound of the formula has binding activity (based on e.g., IC₅₀ or Ki) to D2 and 5HT1B receptors in a ratio of D2: 5HT1B of about 20 or less; in more preferred practices, this ratio is about 10 or less; about 5 or less; most preferably about 1.

Unless otherwise indicated, the term “inhibitory activity” and related variations of same as used herein means that the compound serves, without limitation, as an antagonist, inverse agonist and/or partial agonist (80% antagonism or more) and the like to any of the receptors indicated herein; for example, the compound of Formula I exhibits a binding affinity with a Ki of about 1 micromolar or less, with preferred practices having a Ki of about 100 nanomolar (nM) or less, about 50 nM or less, about 20 nM or less, and most preferably about 10 nM or less, for any of the receptors aforesaid.

As described hereinabove, the second component also includes pharmaceutically acceptable salts of Formula I, e.g. acid addition salts, base addition salts, and prodrugs and solvates thereof. Without limitation, examples of pharmaceutically acceptable acid addition salts of the compounds of Formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malate, di-p-toluoyl tartaric acid, and mandelic acid. Other possible acid addition salts are, e.g., salts containing pharmaceutically acceptable anions, such as the hydroiodide, nitrate, sulfate or bisulfate, phosphate or acid phosphate, acetate, lactate, gluconate, saccharate, benzoate, methanesulfonate, ethanesulfonate, benzenesulfonate, and pamoate (i.e., 1.1′-methylene-bis-(2-hydroxy-3-naphthoate) salts).

The compound of Formula I may have optical centers (e.g., at the 7 and 9a positions indicated) and thus may occur in different enantiomeric configurations. The second component, as defined herein, includes all enantiomers, diastereomers, and other stereoisomers and optical isomers of such compound of Formula I, as well as racemic and other mixtures thereof. For example, the compound of Formula I includes (R) and (S) enantiomers and cis and trans isomers. As defined herein, the second component further includes all radiolabelled forms of the compound of Formula I. Preferred radiolabelled compounds are those wherein the radiolabels are selected from ³H, ¹¹C, ¹⁴C, ¹⁸F, ¹²³I and ¹²⁵I. Such radiolabelled compounds are useful as research and diagnostic tools in metabolism pharmacokinetics studies and in binding assays in animals and man.

In another embodiment, the second component is directed to a compound of Formula I wherein in an assay of D2, 5HT1B or 5HT2A binding said compound exhibits a Ki with intrinsic efficacy of about 1 micromolar or less; preferably exhibiting Ki's of about 100 nanomolar (nM) or less, about 50 nM or less, about 20 nM or less, and most preferably about 10 nM or less. The assays in this regard are those known in or adaptable from the art.

In a preferred embodiment, the compound of Formula I has the formula

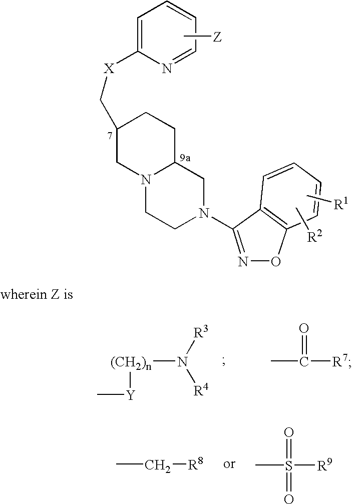

X is oxygen; n is 0; R¹ is hydrogen; R² is hydrogen or halogen; and R³ is hydrogen or a (C₁-C₃)alkyl.

In a more preferred embodiment, Z in the compound of Formula I is 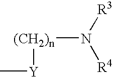
wherein X is oxygen; Y is methylene; n is 0; R¹ is hydrogen; R² is hydrogen or halogen; and R³ is hydrogen or a (C₁-C₃)alkyl. In another preferred embodiment, R² is hydrogen; R³ is hydrogen; and

R⁴ is

a) a (C₁-C₆)alkyl group;

b) a (C₃-C₇)cycloalkyl group; or

c) a 5 to 6 member heterocyclic group, any one of which groups a), b) or c) may be unsubstituted or substituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups.

More preferably, R⁴ is

a) a (C₁-C₄)alkyl which may be unsubstituted or substituted with one of the following: phenyl, cyclopropyl, methoxy, or substituted with a 5 to 6 membered heterocyclic, said heterocyclic having at least one nitrogen or oxygen atom;

b) an unsubstituted (C₃-C₇)cycloalkyl; or

c) a 5 to 6 membered heterocyclic which can be unsubstituted or substituted with a (C₁-C₃)alkyl or a (C₁-C₃)alkoxy, said 5 to 6 member heterocyclic c) having at least one nitrogen atom and up to one other heteroatom selected from nitrogen, oxygen and sulfur. Still more preferably,

R⁴ is

a) an unsubstituted C₄ alkyl; a C₃ alkyl substituted with methoxy; a (C₁-C₂)alkyl substituted with phenyl or cyclopropyl; a (C₁-C₂)alkyl substituted with a 5 membered heterocyclic having a nitrogen or oxygen atom; or a (C₁-C₂)alkyl substituted with a 6 membered heterocyclic having at least one nitrogen;

b) unsubstituted cyclopropyl; or c) a 5 to 6 membered heterocyclic which can be unsubstituted or substituted with a methyl or methoxy, said 5 to 6 membered heterocyclic

c) having at least one nitrogen atom and up to one other heteroatom selected from nitrogen, oxygen and sulfur, said (C₁-C₃)alkyl is methyl and said (C₁-C₃)alkoxy is methoxy.

In another preferred embodiment of the second component, Z is 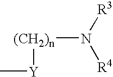
wherein X is oxygen; Y is methylene; n is 0; R³ is (C₁-C₃)alkyl; and R⁴ is a) a (C₁-C₄) alkyl group; or b) a (C₅-C₆)cycloalkyl group, either of which groups a) or b) may be unsubstituted or substituted with one or more (C₁-C₃)alkoxy or amino groups. Preferably, the amino group has the formula —NR⁵R⁶ wherein R⁵ and R⁶ are each independently hydrogen or (C₁-C₄)alkyl; more preferably, R⁴ is a) a (C₁-C₄)alkyl group unsubstituted or substituted with one or more methoxy or amino groups wherein R⁵ is hydrogen and R⁶ is methyl; or b) an unsubstituted (C₅-C₆)cycloalkyl group.

In yet another preferred embodiment in the second component Z is 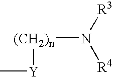
wherein X is oxygen; Y is methylene; n is 0; both R¹ and R² are each hydrogen or halogen; and R³ and R⁴ together with the nitrogen atom to which they are attached form i) a saturated 3 to 7 membered monocyclic ring, said ring i) being unsubstituted or substituted with one or more (C₁-C₄)alkyl, (C₁-C₄)alkoxy(C₁-C₄)alkyl, or hydroxy groups. Alternatively, R³ and R⁴, together with the nitrogen atom to which they are attached, form an unsubstituted 5 to 6 membered ring which has one additional nitrogen, sulfur or oxygen atom. Alternatively, R¹ is hydrogen, R² is halogen; and R⁴ is a) a (C₁-C₅)alkyl; b) a (C₃-C₆) cycloalkyl group, any of which groups a) or b) can be unsubstituted or substituted with one or more of any of the following: cyclopropyl; halogen; hydroxy; a 5 to 6 membered heterocyclic group wherein said 5 to 6 membered heterocyclic group may be unsubstituted or substituted with one or more methyl groups; or phenyl wherein said phenyl may be unsubstituted or substituted with one or more halogens; or R⁴ is c)a 5 member heterocyclic group. Preferably, R² is fluorine; and R³ is hydrogen or methyl.

In still another preferred embodiment of the second component, Z is 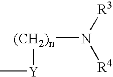
wherein X is oxygen; Y is methylene; n is 0; R¹ is hydrogen, R² is halogen; and R³ and R⁴ together with the nitrogen atom to which they are attached form i) a saturated 3 to 7 membered monocyclic ring, which monocylcic ring may be unsubstituted or substituted with one or more phenyl, (C₁-C₃)alkyl, or (C₁₋₄)alkoxy(C₁₋₄)alkyl groups; or ii) a 5 to 6 membered ring which may be unsubstituted or substituted with one or more (C₁-C₃) alkyl groups, and which has an additional nitrogen atom or oxygen atom.

In the compound of Formula I, in any ring formed by NR³R⁴: (a) there is not more than one ring oxygen atom; (b) no hydroxy, alkoxy, alkoxyalkyl, cyano, amino or alkylamino moiety bonded directly to any ring nitrogen atom; and (c) no ring carbon that is double bonded to another ring carbon and no part of an aromatic ring system can be bonded to a ring oxygen atom or ring nitrogen atom.

Unless otherwise indicated, the following terms and related variations of same as used herein representatively have the meanings ascribed:

“Halogen” and “halo” and the like includes fluoro, chloro, bromo and iodo.

“Alkyl” including as appears in the terms “alkoxy,” “alkoxyalkyl,” and “aralkyl,” includes saturated monovalent hydrocarbon radicals having straight or branched moieties. Examples of alkyl groups include, but are not limited to, methyl, ethyl, n-propyl, isopropyl, and t-butyl.

“Methylene” refers to the divalent radical —(CH₂)ₚ— where p is 1 (methylene), 2 (dimethylene) or 3 (trimethylene).

“Cycloalkyl” includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl; and bicycloalkyl and tricycloalkyl groups that are non-aromatic saturated carbocyclic groups consisting of two or three rings respectively, wherein said rings share at least one carbon atom. For purposes of the present invention, and unless otherwise indicated, bicycloalkyl groups include spiro groups and fused ring groups. Examples of bicycloalkyl groups include, but are not limited to, bicyclo[3.1.0]hexyl, bicyclo[2.2.1]hept-1-yl, norbornyl, spiro[4.5]decyl, spiro[4.4]nonyl, spiro[4.3]octyl, and spiro[4.2]heptyl. An example of a tricycloalkyl group is adamantanyl. Cycloalkyl groups also include groups that are substituted with one or more oxo moieties. Examples of such groups with oxo moieties are oxocyclopentyl and oxocyclobutyl.

“Aryl” includes an organic radical derived from an aromatic hydrocarbon by removal of one hydrogen, such as phenyl, naphthyl, indenyl, indanyl, and fluorenyl; and fused ring groups wherein at least one ring is aromatic.

“Heterocyclic” refers to a cyclic group containing one or more heteroatoms, preferably from one to four heteroatoms, each selected from O, S and N. Heterocyclic groups also include ring systems substituted with one or more oxo moieties. Examples of heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxa-spiro[4.3]octyl, and 1,4-dioxaspiro[4.2]heptyl.

“Heteroaryl” refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, 1,2,3,4-tetrahydroguinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-trizainyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing groups, as derived from the compounds listed above, may be bonded via a C atom or N atom where such is possible. For instance, a group derived from pyrrole may be pyrrol-1-yl (bonded via N) or pyrrol-3-yl (bonded via C). The terms referring to the groups also encompass all possible tautomers.

“Amino” includes moieties of the formula —NR⁵R⁶ wherein R⁵ and R⁶ are each independently hydrogen or (C₁-C₄)alkyl.

Examples of preferred compounds of Formula I are those having the absolute stereochemical configuration defined as (7R, 9aS)-trans or as (7S, 9aS)-cis. Preferred compounds of Formula I include the following:

(7R, 9aS)-trans-Cyclohexyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-2-(Ethyl-{6-[2-(5-fluoro-benzo[d]isoxazol-3-yl)-octahydro-pyrido[1,2-a]pyrazin-7-ylmethoxy]-pyridin-2-ylmethyl}-amino)-ethanol;

(7R, 9aS)-trans-7-[6-(2,6-Dimethyl-piperidin-1-ylmethyl)-pyridin-2-yloxymethyl]-2-(5-fluoro-benzo[d]isoxazol-3-yl)-octahydro-pyrido[1,2-a]pyrazine;

(7R, 9aS)-trans-1-(1-{6-[2-(5-Fluoro-benzo[d]isoxazol-3-yl)-octahydro-pyrido[1,2-a]pyrazin-7-ylmethoxy]-pyridin-2-ylmethyl}-4-phenyl-piperidin-4-yl)ethanone;

(7R, 9aS)-trans-Cyclopropyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-Cyclopropylmethyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-(4-Chloro-benzyl)-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-[2-(1-methyl-pyrrolidin-2-yl)-ethyl]-amine;

(7R, 9aS)-trans-2-(5-Fluoro-benzo[d]isoxazol-3-yl)-7-[6-(4-methyl-piperazin-1-ylmethyl)-pyridin-2-yloxymethyl]-octahydro-pyrido[1,2-a]pyrazine;

(7R, 9aS)-trans-2-(5-Fluoro-benzo[d]isoxazol-3-yl)-7-(6-piperidin-1-ylmethyl-pyridin-2-yloxymethyl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-dimethyl-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(tetrahydro-furan-2-ylmethyl)-amine;

(7R, 9aS)-trans-7-[6-(2,5-Dimethyl-pyrrolidin-1-ylmethyl)-pyridin-2-yloxymethyl]-2-(5-fluoro-benzo[d]isoxazol-3-yl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(2,2,2-trifluoro-ethyl)-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-[3-(4-methyl-piperazin-1-yl)-propyl]-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-pyrrolidin-1-yl-amine;

(7R, 9aS)-trans-7-(6-Azepan-1-ylmethyl-pyridin-2-yloxymethyl)-2-(5-fluoro-benzo[d]isoxazol-3-yl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-(3-methyl-isoxazol-4-yl)-amine;

(7R, 9aS)-trans-(6-(2-Benzo[d]isoxazol-3-yl-octahydro-pyrido[1,2-a]pyrazin-7-ylmethoxy)-pyridin-3-ylmethyl]-thiazol-2-yl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(3-methyl-isoxazol-5-yl)- amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(3-methyl-pyridin-4-yl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(5-methyl-2H-pyrazol-3-yl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(5-methyl-pyridin-2-yl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(1H-pyrazol-3-yl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(6-methyl-pyridin-2-yl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-pyridin-2-yl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-cyclopropylmethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-morpholin-4-yl-ethyl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-pyrimidin-4-yl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-cyclopropyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(6-methoxy-pyridin-3-yl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-pyrrolidin-1-yl-ethyl)-amine;

(7R, 9aS)-trans-2-Benzo[d]isoxazol-3-yl-7-[5-(4-methyl-[1,4]diazepan-1-ylmethyl)-pyridin-2-yloxymethyl]-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-(tetrahydro-furan-2-ylmethyl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(1-phenyl-ethyl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-pyridin-3-ylmethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-pyridin-3-yl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-thiazol-2-yl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-thiazol-2-yl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(3-methyl-pyridin-4-yl)-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(5-methyl-2H-pyrazol-3-yl)-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(5-methyl-pyridin-2-yl)-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(1H-pyrazol-3-yl)-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(6-methyl-pyridin-2-yl)-amine;

(7R, 9aS)-trans-[6-(2-Benzo[d]isoxazol-3-yl-octahydro-pyrido[1,2-a]pyrazin-methoxy)-pyridin-2-ylmethyl]-pyridin-2-yl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-cyclopropylmethyl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(2-morpholin-4-yl-ethyl)-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-pyrimidin-4-yl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(6-methoxy-pyridin-3-yl)-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(2-pyrrolidin-1-yl-ethyl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-diethyl-amine;

(7R, 9aS)-trans-1-[6-(2-Benzo[d]isoxazol-3-yl-octahydro-pyrido[1,2-a]pyrazin-7-ylmethoxy)-pyridin-3-ylmethyl]-pyrrolidin-3-ol;

(7R, 9aS)-trans-1-[6-(2-Benzo[d]isoxazol-3-yl-octahydro-pyrido[1,2-a]pyrazin-7-ylmethoxy)-pyridin-3-ylmethyl]-pyrrolidin-3-ol;

(7S, 9aS)-cis-7-(5-Azetid in-1-ylmethyl-pyridin-2-yloxymethyl)-2-benzo[d]isoxazol-3-yl-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-ethyl)-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-cyclopentyl-methyl-amine;

(7R, 9aS)-trans-N-[6-(2-Benzo[d]isoxazol-3-yl-octahydro-pyrido[1,2-a]pyrazin-7-ylmethoxy)-pyridin-3-ylmethyl]-N,N′-dimethyl-ethane-1,2-diamine;

(7R, 9aS)-trans-2-Benzo[d]isoxazol-3-yl-7-[5-(2-methyl-aziridin-1-ylmethyl)-pyridin-2-yloxymethyl]-octahydro-pyrido[1,2-a]pyrazine;

(7R, 9aS)-trans-7-(5-Azepan-1-ylmethyl-pyridin-2-yloxymethyl)-2-benzo[d]isoxazol-3-yl-octahydro-pyrido[1,2-a]pyrazine;

(7R, 9aS)-trans-2-Benzo[d]isoxazol-3-yl-7-[5-(2-methoxymethyl-pyrrolidin-1-ylmethyl)-pyridin-2-yloxymethyl]-octahydro-pyrido[1,2-a]pyrazine;

(7R, 9aS)-traris-[6-(2-Benzo[d]isoxazol-3-yl-octahydro-pyrido[1,2-a]pyrazin-7-ylmethoxy)-pyridin-3-ylmethyl]-tert-butyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-tert-butyl-methyl-amine;

(7R, 9aS)-trans-2-Benzo[d]isoxazol-3-yl-7-(5-thiazolidin-3-ylmethyl-pyridin-2-yloxymethyl)-octahydro-pyrido[1,2-a]pyrazine;

(7R, 9aS)-trans-2-Benzo[d]isoxazol-3-yl-7-(5-imidazol-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-cyclohexyl-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-dimethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-ethyl-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-1-methyl-ethyl)-amine;

(7R, 9aS)-trans-2-Benzo[d]isoxazol-3-yl-7-[5-(2-methyl-pyrrolidin-1-ylmethyl)-pyridin-2-yloxymethyl]-octahydro-pyrido[1,2-a]pyrazine;

(7S, 9aS)-cis-2-Benzo[d]isoxazol-3-yl-7-(5-piperidin-1-ylmethyl-pyridin-2-yloxymethyl)-octahydro-pyrido[1,2-a]pyrazine;

(7S, 9aS)-cis-[6-(2-Benzo[d]isoxazol-3-yl-octahydro-pyrido[1,2-a]pyrazin-7-ylmethoxy)-pyridin-3-ylmethyl]-dimethyl-amine;

(7S, 9aS)-cis-[6-(2-Benzo[d]isoxazol-3-yl-octahydro-pyrido[1,2-a]pyrazin-7-ylmethoxy)-pyridin-3-ylmethyl]-ethyl-methyl-amine;

(7S, 9aS)-cis-[6-(2-Benzo[d]isoxazol-3-yl-octahydro-pyrido[1,2-a]pyrazin-7-ylmethoxy)-pyridin-3-ylmethyl]-cyclopentyl-methyl-amine;

(7S, 9aS)-cis-[6-(2-Benzo[d]isoxazol-3-yl-octahydro-pyrido[1,2-a]pyrazin-7-ylmethoxy)-pyridin-3-ylmethyl]-(2-methoxy-ethyl)-methyl-amine;

(7S, 9aS)-cis-7-(5-Azetidin-1-ylmethyl-pyridin-2-yloxymethyl)-2-benzo[d]isoxazol-3-yl-octahydro-pyrido[1,2-a]pyrazine;

(7S, 9aS)-cis-2-Benzo[d]isoxazol-3-yl-7-[5-(2-methyl-aziridin-1-ylmethyl)-pyridin-2-yloxymethyl]-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-2-ylmethyl]-(2-methoxy-ethyl)-methyl-amine;

(7R, 9aS)-trans-1-[6-(2-Benzo[d]isoxazol-3-yl-octahydro-pyrido[1,2-a]pyrazin-7-ylmethoxy)-pyridin-2-ylmethyl]-pyrrolidin-3-ol;

(7R, 9aS)-trans-[6-(2-Benzo[d]isoxazol-3-yl-octahydro-pyrido[1,2-a]pyrazin-7-ylmethoxy)-pyridin-2-ylmethyl]-dimethyl-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-cyclohexyl-methyl-amine;

(7S, 9aS)-cis-[6-(2-Benzo[d]isoxazol-3-yl-octahydro-pyrido[1,2-a]pyrazin-7-ylmethoxy)-pyridin-2-ylmethyl]-cyclohexyl-methyl-amine;

(7S, 9aS)-cis-[6-(2-Benzo[d]isoxazol-3-yl-octahydro-pyrido[1,2-a]pyrazin-7-ylmethoxy)-pyridin-2-ylmethyl]-dimethyl-amine;

(7S, 9aS)-cis-[6-(2-Benzo[d]isoxazol-3-yl-octahydro-pyrido[1,2-a]pyrazin-7-ylmethoxy)-pyridin-2-ylmethyl]-(2-methoxy-ethyl)-methyl-amine;

(7R, 9aS)-trans-1-[6-(2-Benzo[d]isoxazol-3-yl-octahydro-pyrido[1,2-a]pyrazin-7-ylmethoxy)-pyridin-2-ylmethyl]-(R)-pyrrolidin-3-ol;

(7R, 9aS)-trans-1-[6-(2-Benzo[d]isoxazol-3-yl-octahydro-pyrido[1,2-a]pyrazin-7-ylmethoxy)-pyridin-2-ylmethyl]-(S)-pyrrolidin-3-ol;

(7S, 9aS)-cis-[6-(2-Benzo[d]isoxazol-3-yl-octahydro-pyrido[1,2-a]pyrazin-7-ylmethoxy)-pyridin-3-ylmethyl]-cyclohexyl-methyl-amine;

(7S, 9aS)-cis-1-[6-(2-Benzo[d]isoxazol-3-yl-octahydro-pyrido[1,2-a]pyrazin-7-ylmethoxy)-pyridin-3-ylmethyl]-pyrrolidin-(S)-3-ol;

(7R, 9aS)-trans-2-Benzo[d]isoxazol-3-yl-7-(5-pyrrolidin-1-ylmethyl-pyridin-2-yloxymethyl)-octahydro-pyrido[1,2-a]pyrazine;

(7R, 9aS)-trans-2-(5-Fluoro-benzo[d]isoxazol-3-yl)-7-(5-pyrrolidin-1-ylmethyl-pyridin-2-yloxymethyl)-octahydro-pyrido[1,2-a]pyrazine;

(7R, 9aS)-trans-1-[6-(2-Benzo[d]isoxazol-3-yl-octahydro-pyrido[1,2-a]pyrazin-7-ylmethoxy)-pyridin-3-ylmethyl]-piperidin-4-ol;

(7S, 9aS)-cis-1-[6-(2-Benzo[d]isoxazol-3-yl-octahydro-pyrido[1,2-a]pyrazin-7-ylmethoxy)-pyridin-3-ylmethyl]-pyrrolidin-(R)-3-ol;

(7R, 9aS)-trans-7-(5-Azetidin-1-ylmethyl-pyridin-2-yloxymethyl)-2-(5-fluoro-benzo[d]isoxazol-3-yl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-3-ylmethyl}-dimethyl-amine;

(7R, 9aS)-trans-Ethyl-{6-[2-(5-fluoro-benzo[d]isoxazol-3-yl)-octahydro-pyrido[1,2-a]pyrazin-7-ylmethoxy]-pyridin-3-ylmethyl}-methyl-amine;

(7R, 9aS)-trans-2-Benzo[d]isoxazol-3-yl-7-(6-pyrrolidin-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-2-ylmethyl]-benzyl-amine;

(7R, 9aS)-trans-2-Benzo[d]isoxazol-3-yl-7-(6-morpholin-4-ylmethyl-pyridin-2-yloxymethyl)-octahydro-pyrido[1,2-a]pyrazine;

(7R, 9aS)-trans-2-Benzo[d]isoxazol-3-yl-7-(5-morpholin-4-ylmethyl-pyridin-2-yloxymethyl)-octahydro-pyrido[1,2-a]pyrazine;

(7R, 9aS)-trans-2-Benzo[d]isoxazol-3-yl-7-(5-piperidin-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-diisopropyl-amine;

(7R, 9aS)-trans-2-Benzo[d]isoxazol-3-yl-7-[5-(4-methyl-piperazin-1-ylmethyl)-pyridin-2-yloxymethyl]-octahydro-pyrido[1,2a]pyrazine;

(7S, 9aS)-cis-2-Benzo[d]isoxazol-3-yl-7-(5-pyrrolidin-1-ylmethyl-pyridin-2-yloxymethyl)-octahydro-pyrido[1,2-a]pyrazine;

(7S, 9aS)-cis-2-Benzo[d]isoxazol-3-yl-7-(6-morpholin-4-ylmethyl-pyridin-2-yloxymethyl)-octahydro-pyrido[1,2-a]pyrazine;

(7S, 9aS)-cis-2-Benzo[d]isoxazol-3-yl-7-(6-pyrrolidin-1-ylmethyl-pyridin-2-yloxymethyl)-octahydro-pyrido[1,2-a]pyrazine;

(7R, 9aS)-trans-2-(5-Fluoro-benzo[d]isoxazol-3-yl)-7-(6-pyrrolidin-1-ylmethyl-pyridin-2-yloxymethyl)-octahydro-pyrido[1,2-a]pyrazine;

(7R, 9aS)-trans-2-(5-Fluoro-benzo[d]isoxazol-3-yl)-7-(6-morpholin-4-ylmethyl-pyridin-2-yloxymethyl)-octahydro-pyrido[1,2-a]pyrazine; and

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(2-morpholin-4-yl-ethyl)-amine.

The most preferred compounds of Formula I are:

(7R, 9aS)-trans-2-Benzo[d]isoxazol-3-yl-7-(6-morpholin-4-ylmethyl-pyridin-2-yloxymethyl)-octahydro-pyrido[1,2-a]pyrazine;

7R, 9aS)-trans-2-Benzo[d]isoxazol-3-yl-7-(5-piperidin-1-ylmethyl-pyridin-2-yloxymethyl)-octahydro-pyrido[1,2-a]pyrazine;

7R, 9aS)-trans-2-(5-Fluoro-benzo[d]isoxazol-3-yl)-7-(5-pyrrolidin-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-diethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-dimethyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-ethyl-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-1-methyl-ethyl)-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-ethyl)-methyl-amine;

(7R, 9aS)-trans-[6-(2-Benzo[d]isoxazol-3-yl-octahydro-pyrido[1,2-a]pyrazin-7-ylmethoxy)-pyridin-3-ylmethyl]-cyclopentyl-methyl-amine;

(7R, 9aS)-trans-7-(5-Azetidin-1-ylmethyl-pyridin-2-yloxymethyl)-2-benzo[d]isoxazol-3-yl-octahydro-pyrido[1,2-a]pyrazine;

(7R, 9aS)-trans-2-Benzo[d]isoxazol-3-yl-7-[5-(2-methyl-aziridin-1-ylmethyl)-pyridin-2-yloxymethyl]-octahydro-pyrido[1,2-a]pyrazine;

(7R, 9aS)-trans-2-Benzo[d]isoxazol-3-yl-7-[5-(2-methoxymethyl-pyrrolidin-1-ylmethyl)-pyridin-2-yloxymethyl]-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-3-ylmethyl]-tert-butyl-amine;

(7S, 9aS)-cis-[6-(2-Benzo[d]isoxazol-3-yl-octahydro-pyrido[1,2-a]pyrazin-7-ylmethoxy)-pyridin-3-ylmethyl]-ethyl-methyl-amine;

(7S, 9aS)-cis-7-(5-Azetidin-1-ylmethyl-pyridin-2-yloxymethyl)-2-benzo[d]isoxazol-3-yl-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-2-ylmethyl]-dimethyl-amine;

(7R, 9aS)-trans-Cyclohexyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-2-(Ethyl-{6-[2-(5-fluoro-benzo[d]isoxazol-3-yl)-octahydro-pyrido[1,2-a]pyrazin-7-ylmethoxy]-pyridin-2-ylmethyl}-amino)-ethanol;

(7R, 9aS)-trans-7-[6-(2,6-Dimethyl-piperidin-1-ylmethyl)-pyridin-2-yloxymethyl]-2-(5-fluoro-benzo[d]isoxazol-3-yl)-octahydro-pyrido[1,2-a]pyrazine;

(7R, 9aS)-trans-(1,2-Dimethyl-propyl)-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-[6-(2-Benzo[d]isoxazol-3-yl-octahydro-pyrido[1,2-a]pyrazin-7-ylmethoxy)-pyridin-2-ylmethyl]-(2-methoxy-ethyl)-methyl-amine;

(7R, 9aS)-trans-1-[6-(2-Benzo[d]isoxazol-3-yl-octahydro-pyrido[1,2-a]pyrazin-7-ylmethoxy)-pyridin-3-ylmethyl]-(S)-pyrrolidin-3-ol;

(7R, 9aS)-trans-1-[6-(2-Benzo[d]isoxazol-3-yl-octahydro-pyrido[1,2-a]pyrazin-7-ylmethoxy)-pyridin-3-ylmethyl]-(R)-pyrrolidin-3-ol;

(7R, 9aS)-trans-2-Benzo[d]isoxazol-3-yl-7-[5-(2-methyl-pyrrolidin-1-ylmethyl)-pyridin-2-yloxymethyl]-octahydro-pyrido[1,2-a]pyrazine;

(7R, 9aS)-trans-1-[6-(2-Benzo[d]isoxazol-3-yl-octahydro-pyrido[1,2-a]pyrazin-7-ylmethoxy)-pyridin-3-ylmethyl]-piperidin-4-ol;

(7R, 9aS)-trans-Cyclopropyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-Cyclopropylmethyl-{6-[2-(5-fluoro-benzo[d]isoxazol-3-yl)-octahydro-pyrido[1,2-a]pyrazin-7-ylmethoxy]-pyridin-2-ylmethyl}-amine;

(7R, 9aS)-trans-2-(5-Fluoro-benzo[d]isoxazol-3-yl)-7-[6-(4-methyl-piperazin-1-ylmethyl)-pyridin-2-yloxymethyl]-octahydro-pyrido[1,2-a]pyrazine;

(7R, 9aS)-trans-2-(5-Fluoro-benzo[d]isoxazol-3-yl)-7-(6-piperidin-1-ylmethyl-pyridin-2-yloxymethyl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-dimethyl-amine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(tetrahydro-furan-2-ylmethyl)-amine;

(7R, 9aS)-trans-7-[6-(2,5-Dimethyl-pyrrolidin-1-ylmethyl)-pyridin-2-yloxymethyl]-2-(5-fluoro-benzo[d]isoxazol-3-yl)-octahydro-pyrido[1,2-a]pyrazine;

(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-[3-(4-methyl-piperazin-1-yl)-propyl]-amine;

(7R, 9aS)-trans-7-(6-Azepan-1-ylmethyl-pyridin-2-yloxymethyl)-2-(5-fluoro-benzo[d]isoxazol-3-yl)-octahydro-pyrido[1,2-a]pyrazinen-2-ylmethyl}-pyrrolidin-1-yl-amine;

(7S, 9aS)-cis-[6-(2-Benzo[d]isoxazol-3-yl-octahydro-pyrido[1,2-a]pyrazin-7-ylmethoxy)-pyridin-3-ylmethyl]-cyclohexyl-methyl-amine;

(7R, 9aS)-trans-1-[6-(2-Benzo[d]isoxazol-3-yl-octahydro-pyrido[1,2-a]pyrazin-7-ylmethoxy)-pyridin-2-ylmethyl]-(S)-pyrrolidin-3-ol;

(7R, 9aS)-trans-1-[6-(2-Benzo[d]isoxazol-3-yl-octahydro-pyrido[1,2-a]pyrazin-7-ylmethoxy)-pyridin-2-ylmethyl]-(R)-pyrrolidin-3-ol;

(7R, 9aS)-trans-2-Benzo[d]isoxazol-3-yl-7-(6-pyrrolidin-1-ylmethyl-pyridin-2-yloxymethyl)-octahydro-pyrido[1,2-a]pyrazine;

(7R, 9aS)-trans-[6-(2-Benzo[d]isoxazol-3-yl-octahydro-pyrido[1,2-a]pyrazin-7-ylmethoxy)-pyridin-2-ylmethyl]-benzyl-amine;

(7R, 9aS)-trans-2-Benzo[d]isoxazol-3-yl-7-(5-pyrrolidin-1-ylmethyl-pyridin-2-yloxymethyl)-octahydro-pyrido[1,2-a]pyrazine; and

(7S, 9aS)-cis-2-Benzo[d]isoxazol-3-yl-7-(5-pyrrolidin-1-ylmethyl-pyridin-2-yloxymethyl)-octahydro-pyrido[1,2-a]pyrazine.

Compounds of Formula I are basic in nature. Thus, the present invention contemplates atypical antipsychotics or salts thereof with pharmaceutically acceptable acid addition salts of Formula I, such as hydrochloride, hydrobromide, hydroiodide, nitrate, sulfate, bisulfate, phosphate, acid phosphate, acetate, lactate, citrate, acid citrate, tartrate, bitrate, succinate, maleate, furmarate, gluconate, saccharate, benzoate, methansulfonate, pamoate, i.e. 1-1′-methylene-bis-(2-hydroxy)-3-naphthoate) salts.

The compounds of Formula I, including the exemplification described hereinabove are prepared as described in copending application Ser. No. 60/453,925, the contents of which are incorporated by reference.

The first component and the second component are present in amounts effective in treating the CNS disorder. More specifically, the selection of the dosage of the first and second component is that which can provide relief to the patient or amelioration of symptoms associated with the disorder or condition of the patient. As is well known, the dosage of each component depends on several factors, such as the potency, the mode of administration, the age and weight of the patient, the severity of the condition to be treated and the like. This is considered to be within the skill of the artisan.

In a preferred embodiment, the molar ratio of the first component to the second component ranges from about 0.1:1.0 to about 1.0:0.1, and even more preferably from about 0.2:1.0 to about 1.0:0.2 and most preferably from about 0.5:1.0 to about 1.0:0.5.

Examples of the dosages of the first component are described in the aforementioned patents or the Physicians Desk Reference, 57^{th} ed., Thompson 2003, which are expressly incorporated by reference.

General outlines of the dosages for examples of atypical antipsychotics and some preferred dosages, are provided herein. This list is not intended to be complete but is merely a guideline for any of the desired combinations of the present invention.

Desirably, when ziprasidone is selected as the first component, the daily dose contains preferably from about 5 mg to about 400 mg of ziprasidone. More preferably, when ziprasidone is the first component, the pharmaceutical composition contains about 20 mg to about 320 mg ziprasidone and even more preferably, each dose contains from about 20 mg to about 160 mg of ziprasidone and most preferably about 20 mg to about 80 mg. More preferably, in this embodiment each dose of the first component contains about 20 mg to about 320 mg of the ziprasidone, even more preferably, each dose contains from about 20 mg to about 160 mg of ziprasidone, and most preferably, each dose contains about 20 mg to about 80 mg of ziprasidone. Pediatric dosages may be less, such as from about 0.5 mg to about 200 mg as determined by a skilled medical practitioner using sound medical judgment. This dosage form permits the full daily dosage to be administered in one or two oral doses, for example.

Olanzapine: from about 0.25 to about 100 mg, once/day; preferably, from about 1 to about 30 mg, once/day; and most preferably about 1 to about 25 mg once/day;

Clozapine: from about 12.5 to about 900 mg daily; preferably, from about 150 to about 450 mg daily;

Risperidone: from about 0.25 to about 16 mg daily; preferably from about 2-8 mg daily;

Sertindole: from about 0.01 to about 1.0 mg/kg daily;

Quetiapine: from about 1.0 to about 40 mg/kg given once daily or in divided doses.

Asenapine: from about 0.005 to about 60 mg total per day, given as a single does or in divided doses.

The compounds of Formula I are present in effective amounts in combination with the antipsychotics. Preferably, the compounds of Formula I are preferably present in the pharmaceutical composition in an amount ranging from about 0.1 to about 200 mg.

The pharmaceutical composition of the present invention may contain just the two components, i.e., the atypical antipsychotic agent and the compound of Formula I. However, it is preferred that they are present with pharmaceutically acceptable carriers. They may be administered in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The pharmaceutical composition of the invention as defined hereinabove may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the pharmaceutical composition may be in powder form for reconstitution with a suitable vehicle, e.g. sterile pyrogen-free water, before use.

In a further aspect, this invention provides pharmaceutical compositions of matter suitable for administration to a human patient as a solution (e.g., as an injectable or intranasally), comprising an inclusion complex of a salt of the compounds of the invention in a material such as cyclodextrin. Either the compound of Formula I or the atypical antipsychotic agent or both may be associated with cyclodextrin. However, it is preferred that the compound of Formula I is associated with cyclodextrin. In a preferred embodiment, said inclusion complex provides an amount of compound of Formula I and the antipsychotic agent of a total of at least 2.5 mgA/ml when the amount of the combination of the compound of Formula I and the antipsychotic agent provided by said complex is measured at a cyclodextrin concentration of 40% w/v in water. The inclusion complex of the compounds in cyclodextrin can first be isolated by drying, usually by lyophilization. The isolated dry inclusion complex can be stored at room temperature for periods up to two years and longer, and reconstituted into a product solution as needed. When a product solution is required, it can be made by dissolving the isolated inclusion complex in water (or other aqueous medium) in an amount sufficient to generate a solution of the required strength for oral or parenteral administration to patients. If parenteral administration is the chosen route of administration, intramuscular injection is preferred.

The compounds may be formulated for fast dispersing dosage forms (fddf), which are designed to release the combinations in the oral cavity. These have often been formulated using rapidly soluble gelatin-based matrices. These dosage forms are well known. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compound of Formula I and the atypical antipsychotic in combination of the invention may also be formulated in rectal compositions such as suppositories or retention enemas, e.g. containing conventional suppository bases such as cocoa butter or other glycerides.

For intranasal administration or administration by inhalation, the compound of Formula I and the atypical antipsychotic invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insufflator may be formulated containing a powder mix of a compound of Formula I and antipsychotic agents and a suitable powder base such as lactose or starch.

A proposed total dose of the active compounds of the invention for oral, parenteral or buccal administration to the average adult human for the treatment of the conditions referred to above (e.g. depression) is about 0.05 to about 200 mg of the combination of the antipsychotic and compound of Formula I per unit dose which could be administered, for example, 1 to 4 times per day.

Aerosol formulations for treatment of the conditions referred to above (e.g. migraine) in the average adult human are preferably arranged so that each metered dose or “puff” of aerosol contains about 20 mg to about 1000 mg of the compound of Formula I and about 1 to about 1000 mg of the atypical antipsychotic (depending upon the atypical antipsychotic selected). The overall daily dose with an aerosol will be within the range of about 100 mg to about 10 mg. Administration may be several times daily, e.g. 2, 3, 4 or 8 times, giving for example, 1, 2 or 3 doses each time.

It is to be noted that the combination of compounds of Formula I and the antipsychotic compound may be administered either alone or in combination with pharmaceutically acceptable carriers by either of the routes previously indicated, and that such administration can be carried out in both single and multiple dosages. More particularly, the active combination can be administered in a wide variety of different dosage forms, i.e. they may be combined with various pharmaceutically-acceptable inert carriers. The oral pharmaceutical formulations can be suitably sweetened and/or flavored by means of various agents of the type commonly employed for such purposes. In general, the compounds of formula I are present in such dosage forms at concentration levels ranging from about 0.5% to about 90% by weight of the total composition, i.e., the compound of Formula I and the atypical antipsychotic, is present in such dosage forms at concentration levels ranging from about 0.5% to about 90% by weight of the total composition, i.e. the total in amounts which are sufficient to provide the desired unit dosage.

The combinations of this invention can also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the patient's condition and requirements.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain an atypical antipsychotic in sustained or controlled release form and, the second therapeutic agent of Formula I in an immediate release form. Alternatively, the atypical antipsychotic can be in immediate release form and the second therapeutic agent of Formula I can be in sustained or controlled release form.

The combinations of this invention can also be administered in parenteral form. For parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, methods of preparing pellets are described in Remington: The Science and Practice of Pharmacy, Mack Publishing Company, Easton, Pa., 19th Edition (1995). Prolonged release pellets are prepared by either coating immediate release pellets or via matrix systems. Coating may be carried out, for example, in coating pans or in fluid bed coater-driers. Extrusion and subsequent spheronization is a long-known method for the preparation of pharmaceutical pellets (J. W. Conine et al., Drug & Cosmetic Ind. 106, 38-41 (1970)). However, other methods such as pelletization may be utilized. Particles may be agglomerated to form spherical granules or pellets, in a high speed mixer granulator, or rotary fluid bed agglomerator. These methods are described by K. W. Olson and A. M. Mehta, lnt.J.Pharm.Tech&.Prod.Mfr. 6 18-24, 1985. Pellets may be also prepared by extrusion of wet masses or melts followed by spheronisation, for example, as described in C. Vervaet, L. Baert & J. P. Remon, Int. J. Pharm. 116, 131-146 (1995)). Excipients used are typically those with plastic qualities such as microcrystalline cellulose, but also mannitol. Small quantities of a polymeric binder are generally added. Surfactants such as sodium dodecyl sulphate or sodium lauryl sulfate may also be incorporated to give easier extrusion.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two different compounds of this invention can be co-administered simultaneously or sequentially in any order, or as a single pharmaceutical composition comprising the antipsychotic agent and the component of Formula I.

Pharmaceutical compositions according to the invention can contain from about 0.1% to about 95% w/w of the combination of therapeutic agents of this invention, preferably about 1% to about 70%. In any event, the composition or formulation to be administered will contain a quantity of therapeutic agent(s) according to the invention in an amount effective to treat the condition or disease of the subject being treated.

The two different compounds of this invention can be co-administered simultaneously or sequentially in any order, or as a single pharmaceutical composition comprising, for example, an atypical psychotic and compound of Formula I as described above.

Since the present invention has an aspect that relates to the treatment of the diseases/conditions described herein with a combination of active ingredients which can be administered separately, the invention also relates to combining separate pharmaceutical compositions in kit form. The kit includes two separate pharmaceutical compositions: the compounds of Formula I and the antipsychotic agent. The kit includes a container for containing the separate compositions such as a divided bottle or a divided foil packet. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent to the skilled practitioner. A “daily dose” can be a single tablet or capsule or several pills or capsules to be taken on a given day.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

It will be understood that while the use of a single atypical antipsychotic as a first component compound is preferred, combinations of two or more atypical antipsychotics may be used as a first component if necessary or desired.

The atypical antipsychotic of the present invention is useful alone or in combination with a second antipsychotic agent, for example, an atypical antipsychotic such as ziprasidone mesylate, or a typical antipsychotic such as haloperidol. It is preferred that if a second antipsychotic agent is used that they both administered to the patient in synergistic effective amounts. It is preferred that the total amount ranges from about 0.0001 to about 1000 mg/kg per day, more preferably from about 0.01 to about 100 mg/kg per day, and most preferably from about 0.1 to about 60 mg/kg per day.

When referring to these preformulation compositions as homogeneous, it is meant that the active ingredients is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 2000 mg of each of the active ingredients of the present invention. Typical unit dosage forms contain from about 1 to about 300 mg, for example about 1, 2, 5, 10, 25, 50 or 100 mg of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The pharmaceutical combinations can be administered on a regimen of up to 6 times per day, preferably 1 to 4 times per day, especially 2 times per day, and most especially once daily.

As used herein the term “subject” includes animals of economic importance such as bovine, ovine, and porcine animals, especially those that produce meat (or poultry), as well as domestic animals (e.g., cats and dogs), sports animals (e.g., horses), zoo animals, and humans, the latter being most preferred.

The combination of a compound of Formula I herein and atypical antipsychotic agent of the present invention are useful for treating CNS diseases, such as schizophrenia, depression and the like.

Administration of the atypical antipsychotic agent in combination with the compound of Formula I in accordance with the present invention, allows a lower dosing of the compound of Formula I to achieve the same antipsychotic affect. The dosage of the compound of Formula I may be reduced by about 25% to about 90% for example, and more preferably by about 40% to about 80% and more preferably from about 50% to about 70%. In fact, the combination may result in synergistic action, wherein the combination results in an enhanced efficacy compared to the psychotropic effect achieved by an independent dose of the atypical anti psychotic.

“Treatment” and “treating” refers to reversing, alleviating, inhibiting the progress of, or preventing the disorder or condition to which such term applies, or one or more symptoms of such condition or disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder. The term “treatment”, as used herein, refers to the act of treating, as “treating” is defined immediately above.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffecctive disorders include disorganized speech, affective flattening, alogia, anhedonia, inappropriate affect, dysphoric mood (in the form of, for example, depression, anxiety or anger), and some indications of cognitive dysfunction.

“Mammal” refers to any member of the class “Mammalia”, including, but not limited to, humans, dogs, and cats.

“Modulating serotonergic neurotransmission” refers to increasing or improving, or decreasing or retarding the neuronal process whereby serotonin is released by a pre-synaptic cell upon excitation and crosses the synapse to stimulate or inhibit the post-synaptic cell.

“Chemical dependency” means an abnormal craving or desire for, or an addiction to a drug. Such drugs are generally taken by the affected individual by any of a variety of means, including oral, parenteral, nasal or by inhalation. Examples of chemical dependencies treatable by the methods of the present invention are dependencies on alcohol, nicotine, cocaine, heroin, phenolbarbitol, and benzodiazepines (e.g. Valium®).

“Treating a chemical dependency” as used herein, means reducing or alleviating such dependency and/or the craving therefore.

The invention provides a method of treating one or more CNS disorders in a mammal, including a human, in need of such treatment. In a preferred practice, the invention can treat one or more CNS disorders with a compound of Formula I and ziprasidone, for example, the invention can be used to treat schizophrenia and depression.

CNS disorders subject of the invention are those known in the art; and include without limitation those wherein a ligand, e.g. an antagonist, an inverse agonist and/or a partial agonist and the like, to D2, 5HT1B, and 5HT2A receptors, either individually or in any combinations thereof, are indicated.

CNS disorders contemplated for treatment by the present invention include, without limitation:

Anxiety or psychotic disorders such as: schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type. Examples of anxiety disorders include, but are not limited to, panic disorder; agoraphobia; a specific phobia; social phobia; obsessive-compulsive disorder; post-traumatic stress disorder; acute stress disorder; and generalized anxiety disorder.

Movement disorders involving: Huntington's disease and dyskinesia associated with dopamine agonist therapy; Parkinson's disease and restless leg syndrome.

Chemical dependencies: for example alcohol, amphetamine, cocaine, opiate, nicotine addiction.

Disorders comprising, as a symptom thereof, a deficiency in cognition: for example, a subnormal functioning in one or more cognitive aspects such as memory, intellect, or learning and logic ability, in a particular individual relative to other individuals within the same general age population. Also, any reduction in any particular individual's functioning in one or more cognitive aspects, for example as occurs in age-related cognitive decline. Examples of disorders that comprise as a symptom a deficiency in cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; Alzheimer's related dementia; delirium; amnestic disorder; post-traumatic stress disorder; mental retardation; a learning disorder, for example reading disorder, mathematics disorder, post operative cognitive decline, or a disorder of written expression; attention-deficit/hyperactivity disorder; and age-related cognitive decline.

Mood disorders or mood episodes such as: major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; treatment resistant depression, SSRI-resistant depression, premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder. Other CNS disorders involved treatment resistant depression, SSR¹ failures, autism and post operative decline.

Other disorders subject to treatment by the invention include those selected from: hypertension, autism, depression (e.g. depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia, amnestic disorders, and age-related cognitive decline (ARCD), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretion), negative symptoms of schizophrenia, schizoaffective disorder, obsessive compulsive disorder, mania, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania and headache (associated with vascular disorders).

The present invention also relates to using the pharmaceutical composition of the present invention for treating cognitive function disorders. As used herein this term “Cognitive function” refers to multiple mental process such as learning perception, language, attention, information processing spatial ability and memory (figural and verbal). The term cognitive function disorder refers to a deficit in one or more of the cognitive functions, e.g., memory functions, problem solving, orientation, and/or abstractions that impinges on an individual's ability to function independently. Examples include dementia, cognitive impairment caused by traumatized brain injury, Alzheimer's diseases, age-related memory disorder, vascular dementia, dementia due to other general medical conditions, e.g., Human Immunodeficiency Virus Infection, head trauma, Parkinson's disease or Huntington's disease, substance-induced dementia, dementia due to multiple etiologies and the like. See, for example, DSM-IV, 4^{th} ed., pp. 135-180.

The present invention also relates to a method for treating a disorder or condition treatable by modulating serotonergic neurotransmission in a mammal, preferably a human, comprising administering to a mammal in need of such treatment a therapeutically effective amount of a compound of component I and component II.

Other disorders and conditions subject to treatment by the present invention are delineated in WO 99/52907 to Bright, the disclosure of which is incorporated herein by reference thereto.

The present invention also relates to a pharmaceutical composition for treating the aforesaid disorders/conditions, among others, comprising a therapeutically effective amount of a compound of the invention, including preferably the compound defined by Formula I and the atypical antipsychotic agent and a pharmaceutically acceptable carrier.

Unless indicated to the contrary, the percentages are by dry weight.

The following examples further illustrate the present invention:

### EXAMPLE 1

A pharmaceutical composition is prepared by combining ziprasidone with (7R,9aS)-trans-2-benzo[d]isoxazol-3-yl-7-(6-morpholin-4-ylmethyl-pyridin-2-yloxymethyl)-octahydro-pyrido[1,2-a]pyrazine in a pharmaceutically acceptable carrier. The composition contains ziprasidone in amounts between about 2 mg to about 200 mg ziprasidone and (7R,9aS)-trans-2-benzo[d]isoxazol-3-yl-7-(6-morpholin-4-ylmethyl-pyridin-2-yloxymethyl)-octahydro-pyrido[1,2-a]pyrazine between about 2 mg to 200 mg. The composition is administered to a patient for the treatment of psychosis associated with Parkinson's disease or subcortical dementias on a daily, twice daily, three times daily, or four times daily basis.

### EXAMPLE 2

| | Quantity | Quantity |
|---|---|---|
| Ingredients | per cap | per batch |
| Ziprasidone HCl | 2-200 mg | 2-200 mg |
| (7R,9aS)-trans-2-benzo-(d)isoxazol-3-yl-7-(6- | 20 mg | 20 mg |
| morphlin-4-ylmethyl-pyridin-2-yl-oxymethyl)- | | |
| octahydro-pyrido (1,2-a)pyrazine | | |
| Methocel E3 | 222 mg | 44 mg |
| Lactose monohydrate | 222 mg | 44 mg |
| Aerosil 10 mg | 10 mg | 2 mg |
| SLS | 10 mg | 2 mg |
| Gl. Acetic acid | q.s. | 40 ml |
| Total weight | 500 mg | |

The ziprasidone is dissolved in the acetic acid. Then the compound of Formula I identified in the chart is dissolved in acetic acid. Lactose, methocel and aerosil are passed through a #40 mesh screen and mix well. The powder blend, is granulated with the drug solution using multiple granulation technique (3-4 times), and the granules are dried at 50° C. The dried granules are passed through a #60 screen and are lubricated with sodium lauryl sulfate (SLS). The powder is filled into capsules.

### EXAMPLE 3

| | Quantity/ |
|---|---|
| Ingredients | Tab |
| Ziprasidone | 20 mg |
| (7R,9aS)-trans-2-benzo[d]isoxazol-3-yl-7-(6- | 2-200 mg |
| morpholin-4-ylmethyl-pyridin-2-yloxymethyl)- | |
| octahydro-pyrido[1,2a]pyrazine | |
| Lactose | 155.5 mg |
| Crosscarmellose sodium (Intra) | 19.5 mg |
| Crosscarmellose sodium (Extra) | 19.5 mg |
| PEG 3000 | 50 mg |
| Aerosil | 6.5 mg |
| Magnesium stearate | 13 mg |
| Povidone | 35 mg |
| Isopropyl alcohol | 0.1 ml |
| Dimethyl sulfoxide | 0.005 ml |
| Total tablet weight | ————— |

(1) The ziprasidone, lactose and crosscarmellose (Intra) are passed through a #60 screen and mixed.

(2) The dimethyl sulfoxide and (7R,9aS)-trans-2-benzo[d]isoxazol-3-yl-7-(6-morpholin-4-ylmethyl-pyridin-2-yloxymethyl)-octahydro-pyrido[1,2-a]pyrazine are heated to form a solution; isopropyl alcohol is added and with continued heating; the PEG 3000 and povidone are added to form a clear solution.

(3) The mass of step 1 is mixed with the solution of step 2 and the resulting product passed through a #20 screen and dry for 30 minutes at 45° C.

(4) It is then passed through a #40 screen and dried again at 45° C.

The product of step 4 is mixed with crosscarmellose (Extra), aerosil and magnesium stearate, and then the granulation is compressed into a tablet.

### EXAMPLE 4

A pharmaceutical preparation is prepared by combining a plurality of pellets, each pellet having an inert core coated with ziprasidone, providing extended release of ziprasidone; and a mixture containing (7R,9aS)-trans-2-benzo[d]isoxazol-3-yl-7-(6-morpholin-4-ylmethyl-pyridin-2-yloxymethyl)-octahydro-pyrido[1,2-a]pyrazine and one or more inert ingredients, wherein the mixture provides extended release of the composition.

The active pellets of ziprasidone are formed by dissolving ziprasidone and ethylcellulose in isopropyl alcohol making a suspension of 25% solid content. The ziprasidone solution is then sprayed onto the inert cores in a fluidized bed processor until the cores are uniformly coated with the desired drug potency. The active core pellets are then dried in a fluidized bed processor until the loss on drying is below 1%. The ziprasidone pellets are then passed through a mesh screen. Methacrylic acid copolymer (Eudragit NE 30D) (30% dispersion) and between 80 are dissolved in a mixture of water and isopropyl alcohol. Talc and magnesium stearate are then dispersed into the solution. The suspension is sprayed onto the ziprasidone pellets in a fluidized bed processor until the loss on drying is less than 1%. The pellets are mixed with talc in a V-blender and passed through a mesh screen. The compound of Formula I mixture is prepared by combining (7R,9aS)-trans-2-benzo[d]isoxazol-3-yl-7-(6-morpholin-4-ylmethyl-pyridin-2-yloxymethyl)-octahydro-pyrido[1,2-a]pyrazine (2-200 mg), xanthan gum, maltodextrin and magnesium stearate in a blender at room temperatures until homogeneous. The ziprasidone pellets are blended with the (7R,9aS)-trans-2-benzo[d]isoxazol-3-yl-7-(6-morpholin-4-ylmethyl-pyridin-2-yloxymethyl)-octahydro-pyrido[1,2-a]pyrazine. The resulting mixture is then placed into capsules.

### EXAMPLE 5

A suspension formulation is prepared by heating water to 70° C. followed by adding methylparaben while stirring at about 200 rpm with an overhead stirrer. After the methylparabens are completely dissolved, the temperature is lowered to about 30° C. The following components are then added in order: xanthan gum, xylitol, anhydrous citric acid, trisodium citrate dihydrate, polysorbate 80, NaCl, ziprasidone hydrochloride, (7R,9aS)-trans-2-Benzo[d]isoxazol-3-yl-7-(6-morpholin-4-ylmethyl-pyridin-2-yloxymethyl)-octahydro-pyrido[1,2-a]pyrazine.

It should be understood that the invention is not limited to the particular embodiments described herein, but that various changes and modifications may be made without departing from the spirit and scope of this novel concept as defined by the following claims.

1. A pharmaceutical composition comprising a therapeutically effective amount of atypical antipsychotic and a compound having the formula 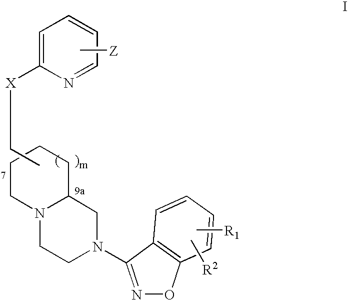
or the (R) or (S) enantiomer thereof, or the cis or trans isomer thereof, or a pharmaceutically acceptable salt, solvate or prodrug thereof, or of any of the foregoing, and a pharmaceutical carrier therefore wherein m is 0 or 1; Z is 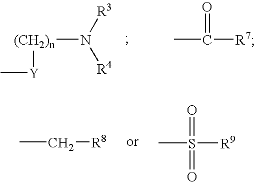
wherein R⁷ is hydrogen or (C₁-C₃)alkoxy; R⁸ is hydrogen, hydroxy, or (C₁-C₃)alkoxy; and R⁹ is (C₁-C₃)alkoxy;
X is oxygen or NR, wherein R is hydrogen or (C₁-C₆)alkyl;
Y is methylene, wherein n is 0, 1 or 2; or oxygen, nitrogen or sulfur, wherein n is 2, 3 or 4;
R¹ and R² are each independently hydrogen, halogen, or a (C₁-C₆)alkyl, (C₁-C₆)alkoxy or a (C₁-C₆)alkoxy(C₁-C₆)alkyl group, any one of which groups may be unsubstituted or substituted with one or more halogens;
R³ and R⁴ are each independently hydrogen, a (C₁-C₆)alkyl, a (C₃-C₇)cycloalkyl, or a 5 to 6 membered heterocyclic group, any one of which groups may be unsubstituted or substituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups; or
R³ and R⁴together with the nitrogen atom to which they are attached form:
a 3 to 7 membered optionally unsaturated monocyclic ring; or
a 4 to 10 membered optionally unsaturated polycyclic ring, wherein said monocyclic or polycyclic ring optionally has one or two additional heteroatoms selected from nitrogen, oxygen and sulfur,
wherein any of said rings (i) or (ii) may be unsubstituted or substituted with one or more (C₁-C₄)alkyl, (C₁C₄)alkoxy, (C₁-C₄)alkoxy(C₁-C₄)alkyl, (C₃-C₇)cycloalkyl, (C₆-C₁₀)aryl, (C₇ to C₁₃)aralkyl, a 5 to 10 membered heteroaryl, hydroxy, amino, cyano, or halogen groups.

2. The pharmaceutical composition according to claim 1 wherein the atypical antipsychotic is ziprasidone, asenapine, olanzapine, clozapine, risperidone, amisulpride, quetiapine, aripiprazole, sertindole, or mirtazapine.

3. The pharmaceutical composition according to claim 2 wherein the antipsychotic is ziprasidone.

4. The pharmaceutical composition according to claim 1 wherein the compound of Formula I has the structure: 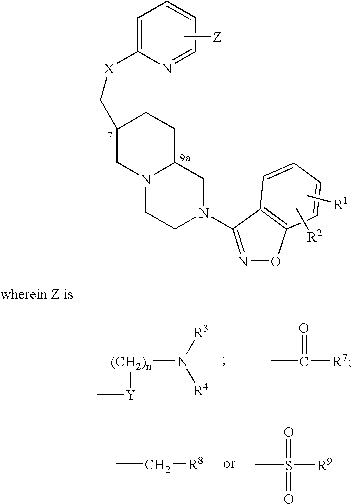
X is oxygen; n is 0; R¹ is hydrogen; R² is hydrogen or halogen; and R³ is hydrogen or a (C₁-C₃)alkyl.

5. The pharmaceutical composition according to claim 4 wherein R² is hydrogen; R³ is hydrogen; and R⁴ is
a) a (C₁-C₆)alkyl group;
b) a (C₃-C₇)cycloalkyl group; or
c) a 5 to 6 member heterocyclic group, wherein any one of which groups a), b) or c) may be unsubstituted or substituted with one or more of any of the following: (C₁-C₄)alkyl, (C₃-C₇)cycloalkyl, (C₁-C₄)alkoxy, (C₆-C₁₀)aryl, a 5 to 6 member heterocyclic, amino, halogen or hydroxy groups.

6. The composition according to claim 5 wherein Z is 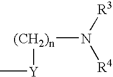
Y is methylene; and R⁴ is
a) a (C₁-C₄)alkyl which may be unsubstituted or substituted with one of the following: phenyl, cyclopropyl, methoxy, or substituted with a 5 to 6 membered heterocyclic, said heterocyclic having at least one nitrogen or oxygen atom;
b) an unsubstituted (C₃-C₇)cycloalkyl; or
c) a 5 to 6 membered heterocyclic which can be unsubstituted or substituted with a (C₁-C₃)alkyl or a (C₁-C₃)alkoxy, said 5 to 6 member heterocyclic c) having at least one nitrogen atom and up to one other heteroatom selected from nitrogen, oxygen and sulfur.

7. The composition according to claim 6 wherein R⁴ is
a) an unsubstituted C₄ alkyl; a C₃ alkyl substituted with methoxy; a (C₁-C₂)alkyl substituted with phenyl or cyclopropyl; a (C₁-C₂)alkyl substituted with a 5 membered heterocyclic having a nitrogen or oxygen atom; or a (C₁-C₂)alkyl substituted with a 6 membered heterocyclic having at least one nitrogen;
b) unsubstituted cyclopropyl; or
c) a 5 to 6 membered ring which can be unsubstituted or substituted with a methyl or methoxy, said 5 to 6 membered ring c) having at least one nitrogen atom and up to one other heteroatom selected from nitrogen, oxygen and sulfur, said (C₁-C₃)alkyl is methyl and said (C₁-C₃)alkoxy is methoxy.

8. The composition according to claim 4 wherein R² is hydrogen; R³ is (C₁-C₃)alkyl; and R⁴ is
a) a (C₁-C₄)alkyl group; or
b) a (C₅-C₆)cycloalkyl group, either of which groups a) or b) may be unsubstituted or substituted with one or more (C₁-C₃)alkoxy or amino groups.

9. The composition according to claim 1 wherein R⁴ is
a) a (C₁-C₄)alkyl group unsubstituted or substituted with one or more methoxy or amino groups wherein R⁵ is hydrogen and R⁶ is methyl; or
b) an unsubstituted (C₅-C₆)cycloalkyl group.

10. The composition according to claim 1 wherein Z is 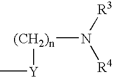
wherein Y is methylene; X is oxygen; n is 0; R¹ is hydrogen; R² is hydrogen; and R³ and R⁴ together with the nitrogen atom to which they are attached form i) a saturated non-aromatic 3 to 7 membered monocyclic ring, said ring i) being unsubstituted or substituted with one or more (C₁-C₄)alkyl, (C₁-C₄)alkoxy(C₁-C₄)alkyl, or hydroxy groups.

11. The composition according to claim 1 wherein Z is 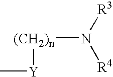
wherein Y is methylene; wherein X is oxygen; n is 0; R¹ is hydrogen; R² is hydrogen; and R³ and R⁴ together with the nitrogen atom to which they are attached form iii) an unsubstituted 5 to 6 membered heterocyclic ring, which heterocyclic ring, in addition to the nitrogen atom to which R³ and R⁴ are attached, has one additional nitrogen atom, or one sulfur or one oxygen atom.

12. The composition according to claim 4 wherein Z is 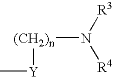
wherein Y is methylene; n is 0; R² is halogen; and R⁴ is
a) a (C₁-C₅ )alkyl;
b) a (C₃-C₆) cycloalkyl group, any of which groups a) or b) can be unsubstituted or substituted with one or more of any of the following: cyclopropyl; halogen; hydroxy; a 5 to 6 membered heterocyclic group wherein said 5 to 6 membered heterocyclic group may be unsubstituted or substituted with one or more methyl groups; or phenyl wherein said phenyl may be unsubstituted or substituted with one or more halogens; or R⁴ is
c) a 5 member heterocyclic group.

13. The composition according to claim 1 wherein said compound of Formula I is selected from the group consisting of:
(7R, 9aS)-trans-2-Benzo[d]isoxazol-3-yl-7-(6-morpholin-4-ylmethyl-pyridin-2-yloxymethyl)-octahydro-pyrido[1,2-a]pyrazine;
(7R, 9aS)-trans-2-Benzo[d]isoxazol-3-yl-7-(5-piperidin-1-ylmethyl-pyridin-2-yloxymethyl)-octahydro-pyrido[1,2-a]pyrazine
(7R, 9aS)-trans-2-(5-Fluoro-benzo[d]isoxazol-3-yl)-7-(5-pyrrolidin-1-ylmethyl-pyridin-2-yloxymethyl)-octahydro-pyrido[1,2-a]pyrazine
(7R, 9aS)-trans-[6-(2-Benzo[d]isoxazol-3-yl-octahydro-pyrido[1,2-a]pyrazin-7-ylmethoxy)-pyridin-3-ylmethyl]-diethyl-amine
(7R, 9aS)-trans-[6-(2-Benzo[d]isoxazol-3-yl-octahydro-pyrido[1,2-a]pyrazin-7-ylmethoxy)-pyridin-3-ylmethyl]-dimethyl-amine
(7R, 9aS)-trans-[6-(2-Benzo[d]isoxazol-3-yl-octahydro-pyrido[1,2-a]pyrazin-7-ylmethoxy)-pyridin-3-ylmethyl]-ethyl-methyl-amine
(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-1-methyl-ethyl)-amine
(7R, 9aS)-trans-[6-(2-Benzo[d]isoxazol-3-yl-octahydro-pyrido[1,2-a]pyrazin-7-ylmethoxy)-pyridin-3-ylmethyl]-(2-methoxy-ethyl)-methyl-amine
(7R, 9aS)-trans-[6-(2-Benzo[d]isoxazol-3-yl-octahydro-pyrido[1,2-a]pyrazin-7-ylmethoxy)-pyridin-3-ylmethyl]-cyclopentyl-methyl-amine
(7R, 9aS)-trans-7-(5-Azetidin-1-ylmethyl-pyridin-2-yloxymethyl)-2-benzo[d]isoxazol-3-yl-octahydro-pyrido[1,2-a]pyrazine
(7R, 9aS)-trans-2-Benzo[d]isoxazol-3-yl-7-[5-(2-methyl-aziridin-1-ylmethyl)-pyridin-2-yloxymethyl]-octahydro-pyrido[1,2-a]pyrazine
(7R, 9aS)-trans-2-Benzo[d]isoxazol-3-yl-7-[5-(2-methoxymethyl-pyrrolidin-1-ylmethyl)-pyridin-2-yloxymethyl]-octahydro-pyrido[1,2-a]pyrazine
(7R, 9aS)-trans-[6-(2-Benzo[d]isoxazol-3-yl-octahydro-pyrido[1,2-a]pyrazin-7-ylmethoxy)-pyridin-3-ylmethyl]-tert-butyl-amine
(7S, 9aS)-cis-[6-(2-Benzo[d]isoxazol-3-yl-octahydro-pyrido[1,2-a]pyrazin-7-ylmethoxy)-pyridin-3-ylmethyl]-ethyl-methyl-amine
(7S, 9aS)-cis-7-(5-Azetidin-1-ylmethyl-pyridin-2-yloxymethyl)-2-benzo[d]isoxazol-3-yl-octahydro-pyrido[1,2-a]pyrazine
(7R, 9aS)-trans-[6-(2-Benzo[d]isoxazol-3-yl-octahydro-pyrido[1,2-a]pyrazin-7-ylmethoxy)-pyridin-2-ylmethyl]-dimethyl-amine
(7R, 9aS)-trans-Cyclohexyl-{6-[2-(5-fluoro-benzo[d]isoxazol-3-yl)-octahydro-pyrido[1,2-a]pyrazin-7-ylmethoxy]-pyridin-2-ylmethyl}-amine
(7R, 9aS)-trans-2-(Ethyl-{6-[2-(5-fluoro-benzo[d]isoxazol-3-yl)-octahydro-pyrido[1,2-a]pyrazin-7-ylmethoxy]-pyridin-2-ylmethyl}-amino)-ethanol
(7R, 9aS)-trans-7-[6-(2,6-Dimethyl-piperidin-1-ylmethyl)-pyridin-2-yloxymethyl]-2-(5-fluoro-benzo[d]isoxazol-3-yl)-octahydro-pyrido[1,2-a]pyrazine
(7R, 9aS)-trans-(1,2-Dimethyl-propyl)-{6-[2-(5-fluoro-benzo[d]isoxazol-3-yl)-octahydro-pyrido[1,2-a]pyrazin-7-ylmethoxy]-pyridin-2-ylmethyl}-amine
(7R, 9aS)-trans-[6-(2-Benzo[d]isoxazol-3-yl-octahydro-pyrido[1,2-a]pyrazin-7-ylmethoxy)-pyridin-2-ylmethyl]-(2-methoxy-ethyl)-methyl-amine
(7R, 9aS)-trans-1-[6-(2-Benzo[d]isoxazol-3-yl-octahydro-pyrido[1,2-a]pyrazin-7-ylmethoxy)-pyridin-3-ylmethyl]-(S)-pyrrolidin-3-ol
(7R, 9aS)-trans-1-[6-(2-Benzo[d]isoxazol-3-yl-octahydro-pyrido[1,2-a]pyrazin-7-ylmethoxy)-pyridin-3-ylmethyl]-(R)-pyrrolidin-3-ol
(7R, 9aS)-trans-2-Benzo[d]isoxazol-3-yl-7-[5-(2-methyl-pyrrolidin-1-ylmethyl)-pyridin-2-yloxymethyl]-octahydro-pyrido[1,2-a]pyrazine
(7R, 9aS)-trans-1-[6-(2-Benzo[d]isoxazol-3-yl-octahydro-pyrido[1,2-a]pyrazin-7-ylmethoxy)-pyridin-3-ylmethyl]-piperidin-4-ol
(7R, 9aS)-trans-Cyclopropyl-{6-[2-(5-fluoro-benzo[d]isoxazol-3-yl)-octahydro-pyrido[1,2-a]pyrazin-7-ylmethoxy]-pyridin-2-ylmethyl}-amine
(7R, 9aS)-trans-Cyclopropylmethyl-{6-[2-(5-fluoro-benzo[d]isoxazol-3-yl)-octahydro-pyrido[1,2-a]pyrazin-7-ylmethoxy]-pyridin-2-ylmethyl}-amine
(7R, 9aS)-trans-2-(5-Fluoro-benzo[d]isoxazol-3-yl)-7-[6-(4-methyl-piperazin-1-ylmethyl)-pyridin-2-yloxymethyl]-octahydro-pyrido[1,2-a]pyrazine
(7R, 9aS)-trans-2-(5-Fluoro-benzo[d]isoxazol-3-yl)-7-(6-piperidin-1-ylmethyl-pyridin-2-yloxymethyl)-octahydro-pyrido[1,2-a]pyrazine
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-dimethyl-amine
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-(tetrahydro-furan-2-ylmethyl)-amine
(7R, 9aS)-trans-7-[6-(2,5-Dimethyl-pyrrolidin-1-ylmethyl)-pyridin-2-yloxymethyl]-2-(5-fluoro-benzo[d]isoxazol-3-yl)-octahydro-pyrido[1,2-a]pyrazine
(7R, 9aS)-trans-{6-[2-(5-Fluoro-benzo[d]isoxazol-3-yl)-octahydro-pyrido[1,2-a]pyrazin-7-ylmethoxy]-pyridin-2-ylmethyl}-[3-(4-methyl-piperazin-1-yl)-propyl]-amine
(7R, 9aS)-trans-{6-[2-(5-Fluoro-benzo[d]isoxazol-3-yl)-octahydro-pyrido[1,2-a]pyrazin-7-ylmethoxy]-pyridi(7R, 9aS)-trans-7-(6-Azepan-1-ylmethyl-pyridin-2-yloxymethyl)-2-(5-fluoro-benzo[d]isoxazol-3-yl)-octahydro-pyrido[1,2-a]pyrazinen-2-ylmethyl}-pyrrolidin-1-yl-amine
(7S, 9aS)-cis-[6-(2-Benzo[d]isoxazol-3-yl-octahydro-pyrido[1,2-a]pyrazin-7-ylmethoxy)-pyridin-3-ylmethyl]-cyclohexyl-methyl-amine
(7R, 9aS)-trans-1-[6-(2-Benzo[d]isoxazol-3-yl-octahydro-pyrido[1,2-a]pyrazin-7-ylmethoxy)-pyridin-2-ylmethyl]-(S)-pyrrolidin-3-ol
(7R, 9aS)-trans-1-[6-(2-Benzo[d]isoxazol-3-yl-octahydro-pyrido[1,2-a]pyrazin-7-ylmethoxy)-pyridin-2-ylmethyl]-(R)-pyrrolidin-3-ol
(7R, 9aS)-trans-2-Benzo[d]isoxazol-3-yl-7-(6-pyrrolidin-1-ylmethyl-pyridin-2-yloxymethyl)-octahydro-pyrido[1,2-a]pyrazine
(7R, 9aS)-trans-[6-(2-Benzo[d]isoxazol-3-yl-octahydro-pyrido[1,2-a]pyrazin-7-ylmethoxy)-pyridin-2-ylmethyl]-benzyl-amine
(7R, 9aS)-trans-2-Benzo[d]isoxazol-3-yl-7-(5-pyrrolidin-1-ylmethyl-pyridin-2-yloxymethyl)-octahydro-pyrido[1,2-a]pyrazine; and
(7S, 9aS)-cis-2-Benzo[d]isoxazol-3-yl-7-(5-pyrrolidin-1-ylmethyl-pyridin-2-yloxymethyl)-octahydro-pyrido[1,2-a]pyrazine.

14. A method for treating one or more central nervous system disorders comprising administering to a mammal in need of such treatment a therapeutically effective amount of the composition of claim 1.

15. A method of treating a disorder selected from the group consisting of schizophrenia, schizophreniform disorder, schizoaffective disorder, delusional disorder; substance-induced psychotic disorder, personality disorder of the paranoid type, personality disorder of the schizoid type, panic disorder, phobias, obsessive-compulsive disorder, stress disorders, generalized anxiety disorder, movement disorders involving Huntington's disease, dyskinesia associated with dopamine agonist therapy, Parkinson's disease, restless leg syndrome, chemical dependencies, disorders comprising as a symptom thereof a deficiency in cognition, dementias, mood disorders and episodes in a mammal; anxiety or psychotic disorders including schizophrenia, of the paranoid, disorganized, catatonic, undifferentiated, or residual type; schizophreniform disorder; schizoaffective disorder of the delusional type or the depressive type; delusional disorder; psychosis induced by alcohol, amphetamine, cannabis, cocaine, hallucinogens, inhalants, opioids, or phencyclidine; personality disorder of the paranoid type; and personality disorder of the schizoid type, panic disorder; agoraphobia; a specific phobia; social phobia; obsessive-compulsive disorder; post-traumatic stress disorder; acute stress disorder; chemical dependencies: for alcohol, amphetamine, cocaine, opiate, nicotine addiction; disorders comprising, as a symptom thereof, a deficiency in cognition, a subnormal functioning in one or more cognitive aspects; deficiency in memory, intellect, or learning and logic ability, in a particular individual relative to other individuals within the same general age population; reduction in any particular individual's functioning in one or more cognitive aspects, age-related cognitive decline; dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; Alzheimer's related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline; mood disorders or mood episodes; major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder; delusional disorder or schizophrenia; a bipolar disorder, bipolar I disorder, bipolar II disorder, and cyclothymic disorder, disorders subject to treatment by inhibition of any or all of the D2, 5HT1B, and 5HT2A receptors: hypertension, depression; depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression, generalized anxiety disorder, phobias; agoraphobia, social phobia and simple phobias, posttraumatic stress syndrome, avoidant personality disorder, premature ejaculation, eating disorders; anorexia nervosa and bulimia nervosa, obesity, chemical dependencies, addictions to alcohol, cocaine, heroin, phenobarbital, nicotine and benzodiazepines, cluster headache, migraine, pain, Alzheimer's disease, obsessive-compulsive disorder, panic disorder, memory disorders; dementia, amnestic disorders, and age-related cognitive decline (ARCD), Parkinson's diseases; dementia in Parkinson's disease, neuroleptic-induced parkinsonism and tardive dyskinesias, endocrine disorders; hyperprolactinaemia, vasospasm, vasospasm in the cerebral vasculature, cerebellar ataxia, gastrointestinal tract disorders involving changes in motility and secretion, negative symptoms of schizophrenia, schizoaffective disorder, obsessive compulsive disorder, mania, premenstrual syndrome, fibromyalgia syndrome, stress incontinence, Tourette's syndrome, trichotillomania, kleptomania, male impotence, cancer; small cell lung carcinoma, chronic paroxysmal hemicrania and headache associated with vascular disorders comprising administering to a mammal in need of such treatment a therapeutically effective amount of the composition of claim 1.
